(19) United States
(12) Patent Application Publication
(10) Pub. No.: US 2012/0220895 A1
(43) Pub. Date: Aug. 30, 2012

(54) ALIGNMENT MECHANISM FOR CARTRIDGE-BASED DEVICES

(75) Inventors: Doug VINE, Alpharetta, GA (US); Gil KAN, Atlanta, GA (US)

(73) Assignee: FACET TECHNOLOGIES, LLC, Kennesaw, GA (US)

(21) Appl. No.: 13/405,859

(22) Filed: Feb. 27, 2012

Related U.S. Application Data
(60) Provisional application No. 61/446,649, filed on Feb. 25, 2011.

(51) Int. Cl. A61B 5/151

(52) U.S. Cl. 600/583

(57) ABSTRACT

A lancing device and a cartridge of lancets are operable to sequentially advance each lancet to an active position for use in sampling a bodily fluid. An alignment mechanism precisely aligns the lancets in the active position to promote smooth operation and prevent jamming. The alignment mechanism includes at least one register element and at least one spring-biased element movable between engaged and retracted positions. In an example embodiment, one spring-biased arm extends from the lancing-device housing and includes a protrusion, and a series of recessed register surfaces are defined in a carrier for the lancets. In the engaged position, the spring-biased protrusion fully mates with the register recess under the spring-biasing influence to precisely align the active lancet. And in the retracted position, the spring-biased protrusion is deflected from engagement with the register recess as the carrier is advanced to advance a fresh lancet into the active position.

### CROSS-REFERENCE TO RELATED APPLICATION

This application claims the priority benefit of U.S. Provisional Patent Application Ser. No. 61/446,649 filed Feb. 25, 2011, the entirety of which is hereby incorporated herein by reference for all purposes.

### TECHNICAL FIELD

The present invention relates generally to cartridge-based devices, and more particularly, to mechanisms for precisely aligning medical items to be sequentially advanced for use in, or dispensing from, cartridges or other dispensers.

### BACKGROUND

Cartridge-based devices can be utilized to dispense or apply certain medical items, such as medicine, lancets, sutures, needles, surgical staples, etc., or other medical or other items. For example, a replaceable cartridge containing a plurality of items for sequential use may be loaded into a tool or other device. Such devices can be manufactured to interface with humans directly or with machines utilizing the items therein. For example, many medical procedures require puncturing of the skin, and sometimes underlying tissues, of an animal or human subject. A sharp lancet tip is commonly used to puncture the subject's skin at a lancing site to obtain a sample of blood, interstitial fluid, or other body fluid, as for example in blood-glucose monitoring by diabetics and in blood-typing and blood-screening applications.

In some situations, a person must periodically sample their blood for multiple testing throughout the day or week. This is typically done using a lancing device of some sort. Because re-use of a lancet can result in infection or spread of blood-borne contaminants, persons requiring repeated testing often must carry multiple lancets with them, with each lancet separately loaded into the lancing device for each sampling. This can be inconvenient and may lead to reduced compliance with a prescribed test regimen.

Cartridge-type lancing devices have been developed to allow the user to load cartridges into the lancing device, each cartridge holding multiple lancets for sequential use. These cartridge-type lancing devices typically operate by sequentially advancing each of the lancets in the cartridge for use, charging a drive spring, and, upon actuation of an actuator, releasing the lancet to be propelled by the discharging drive spring through a lancing stroke. The lancets are sequentially advanced into an active position for use by an advancing mechanism. However, on occasion the advancing mechanism can advance a new lancet slightly too far or not quite far enough, which can sometimes result in the device becoming jammed and inoperable until the jam is cleared. This can be a tolerances issue resulting from the advancing mechanism including mechanical parts (e.g., gears) that are manufactured precisely but with certain acceptable tolerances, or this can be the result of slippage or binding in some designs. As such, existing cartridge-type lancing devices have not proven entirely satisfactory in their ease-of-use, cost, and/or reliability.

Accordingly, it can be seen that needs exist for improvements in advancement mechanisms for cartridge-based devices. It is to the provision of an improved cartridge-based device and cartridge meeting these and other needs that the present invention is primarily directed.

### SUMMARY

The present invention relates to an improvement in cartridge-based lancing devices that promotes smooth lancet operation and prevents lancet jamming. A lancing device and a cartridge of lancets are operable to sequentially advance each lancet to an active position for use in sampling a bodily fluid. An alignment mechanism precisely aligns the lancets in the active position to promote smooth operation and prevent jamming. The alignment mechanism includes at least one register element and at least one spring-biased element movable between engaged and retracted positions. In an example embodiment, one spring-biased arm extends from the lancing-device housing and includes a protrusion, and a series of recessed register surfaces are defined in a carrier for the lancets. In the engaged position, the spring-biased protrusion fully mates with the register recess under the spring-biasing influence to precisely align the active lancet. And in the retracted position, the spring-biased protrusion is deflected from engagement with the register recess as the carrier is advanced to advance a fresh lancet into the active position.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a top perspective view of a cartridge-based lancing device according to an example embodiment of the present invention, showing an operating handle in a retracted position.

FIG. 2 shows the lancing device of FIG. 1 with the operating handle in an extended position.

FIG. 3 is a bottom perspective view of the lancing device of FIG. 1.

FIG. 4 is a top view of the lancing device of FIG. 2, with the device's top housing portion and interior base wall removed to reveal major internal components of an advancement mechanism.

FIG. 5 is a top perspective view of the lancing device portion of FIG. 4, with the interior base wall shown concealing the major internal components of the advancement mechanism.

FIG. 6 shows the lancing device portion of FIG. 5, with a multi-lancet cartridge installed, the cartridge's top housing portion removed, and the lancets not shown.

FIG. 7 is a bottom view of the multi-lancet cartridge portion of FIG. 6, with the cartridge's bottom housing portion removed to reveal the lancets in place under the lancet carrier.

FIG. 8 is a side view of the multi-lancet cartridge of FIG. 7, showing portions of a lancet-alignment mechanism.

FIG. 9 is a cross-sectional detail view of a portion of the multi-lancet cartridge of FIG. 8 taken at line 9-9.

FIG. 10 shows the lancing device of FIG. 8, with the lancet-alignment mechanism in operation.

FIG. 11 is a top view of the lancing device of FIG. 8, with the lancet tip in its extended position.

FIG. 12 is a side view of a portion of the lancet-alignment mechanism of the lancing device of FIG. 8, showing a resilient arm in mating engagement with a recess of the lancet carrier to precisely align the carrier, and thus a fresh active lancet, in the active position.

FIG. 13 shows the lancet-alignment mechanism portion of FIG. 12, with the resilient arm deflected/retracted from the recess and sliding across the advancing carrier as the now-used lancet is being advanced out of, and a next fresh lancet is being advanced into, the active position.

FIG. 14 shows the lancet-alignment mechanism portion of FIG. 13, with the resilient arm biasing into mating engagement with the recess to precisely align the next fresh lancet in the active position of FIG. 12.

### DETAILED DESCRIPTION OF EXAMPLE EMBODIMENTS

The present invention may be understood more readily by reference to the following detailed description of example embodiments taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be unnecessarily limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

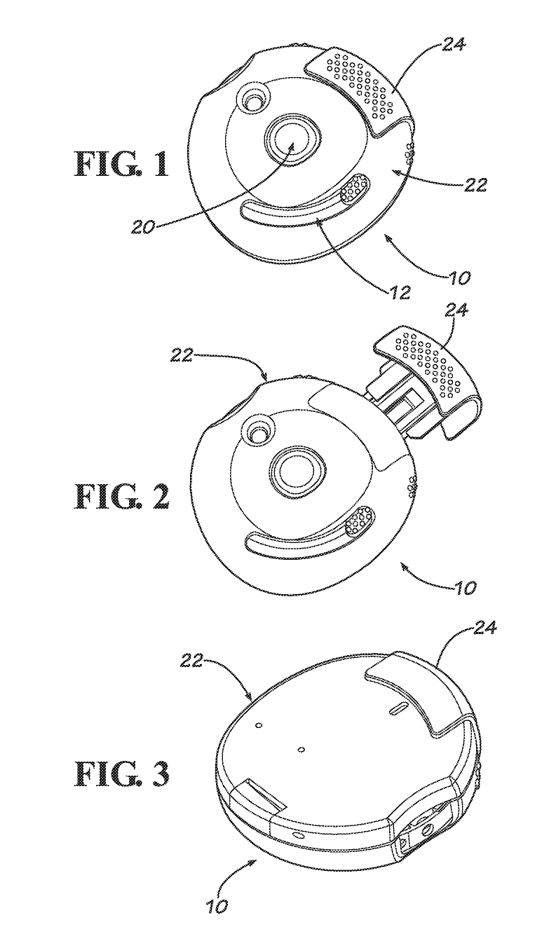

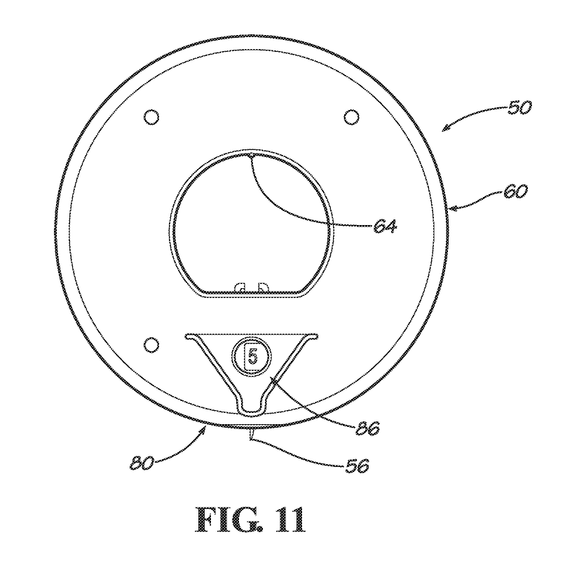

With reference now to the drawing figures, wherein like reference numbers represent corresponding parts throughout the several views, FIGS. 1-14 show a lancing device 10 according to an example embodiment of the present invention. The lancing device 10 is used with a cartridge 50 holding a plurality of lancets 52 each having a body 54, a sharp puncturing tip 56, and a sterility cap 58.

It will be understood that the term “cartridge” as used herein includes carrousels, clips, and other types of dispensers of lancets or other items, whether they are replaceable cartridges used with re-useable lancing devices or whether they are integral to and disposable with the lancing devices after all the lancets have been used once, and whether the lancets or other items are rotationally advanced, linearly advanced, or otherwise configured in the cartridges. In addition, it will be understood that instead of a lancing device used with a cartridge of lancets, the herein-described assemblies and aspects of the invention can be embodied in other medical devices for using or dispensing other items, construction tools for using or dispensing fasteners or other items, firearms for using or dispensing ammunition or other items, food devices for using or dispensing candy or other food items, or other devices using cartridges holding other items that are advanced for use or dispensing. For example, the assemblies and aspects of the invention described herein can be adapted for use in medical devices that dispense sutures, needles, medicines, or surgical staples.

The components of the lancing device 10 and cartridge 50 can be fabricated from conventional materials using conventional fabrication techniques. Thus, these components can be made of plastics, metals, or other suitable materials known in the art, by molding, machining, stamping or other suitable processes known in the art.

The lancing device 10 of the depicted embodiment includes a depth-adjustment mechanism 12, an advancement mechanism 14, a lancet cap-displacement mechanism 16, a charging mechanism 18, and an actuation mechanism 20. The depth-adjustment mechanism 12 operates to provide adjustment for different puncturing depths of the lancet tip 56 into the user's skin during a lancing stroke. The advancement mechanism 14 operates to sequentially advance the lancets 52 to an active position for use. The cap-displacement mechanism 16 operates to remove the sterility caps 58 from the lancet tips 56 before the lancing stroke. The charging mechanism 18 operates to retract an active-positioned lancet 52 and charge a drive spring. And the actuation mechanism 20 operates to release the lancet from the charged position so that it can be propelled by the discharging drive spring through the lancing stroke. Details of the structure, manufacture, and operation of these mechanisms are included in the disclosure of U.S. patent application Ser. No. 12/892,324, filed Sep. 28, 2010, which is hereby incorporated by reference herein.

In various embodiments not expressly disclosed herein but included within the scope of the present invention, the lancing device and/or cartridge can be provided with additional or alternative operational mechanisms to perform the same or other basic functions required for advancing lancets or other items, they can be provided without some of these mechanisms, or they can be provided with some combination of these and/or other mechanisms. As such, included within the scope of the present invention are embodiments in which the cartridge, and the device the cartridge is used with, include operational mechanisms other than those expressly described herein.

Referring to FIGS. 1-3, the lancing device 10 includes a housing 22 and an operating handle 24. The housing 12 can include a top portion and a bottom portion that can be at least partially separable (e.g., pivotally in a clamshell arrangement) from a closed position to an open position for replacing the lancet cartridges. The operating handle 24 is translationally mounted to the housing 22 so that it slides between a retracted position (see FIG. 1) and an extended position (see FIG. 2). The operating handle 24 includes a grip portion for a user to grasp to push and pull the handle between the retracted and extended positions. And the operating handle 24 and the housing 22 have cooperating stop surfaces that abut each other to limit the translating travel of the operating handle 24 between the retracted and extended positions.

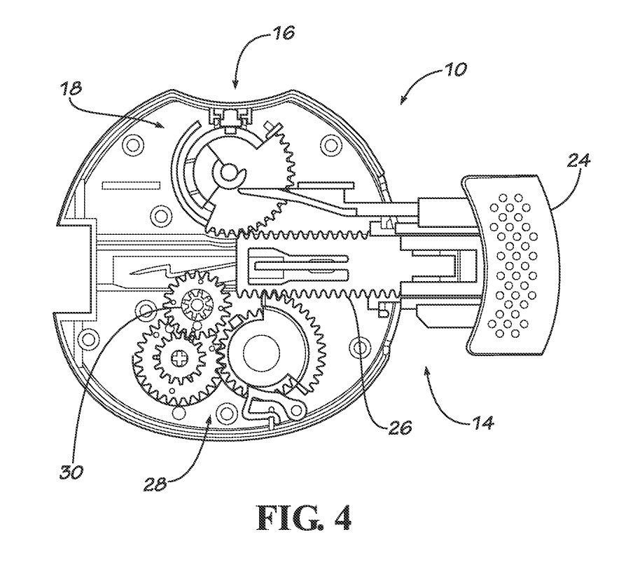

Referring to FIGS. 4-5, the advancement mechanism 14 is housed within the lancing-device housing 22. In the depicted embodiment, the advancement mechanism 14 includes an advancement gear assembly 28 including an advancement handle gear 26 on the handle 24, one or more intermediate gears driven by the advancement handle gear, and a pinion gear 30 driven by one or more of the intermediate gears. As noted, additional details of the structure and operation of the advancement mechanism 14 are disclosed in the incorporated-by-reference application.

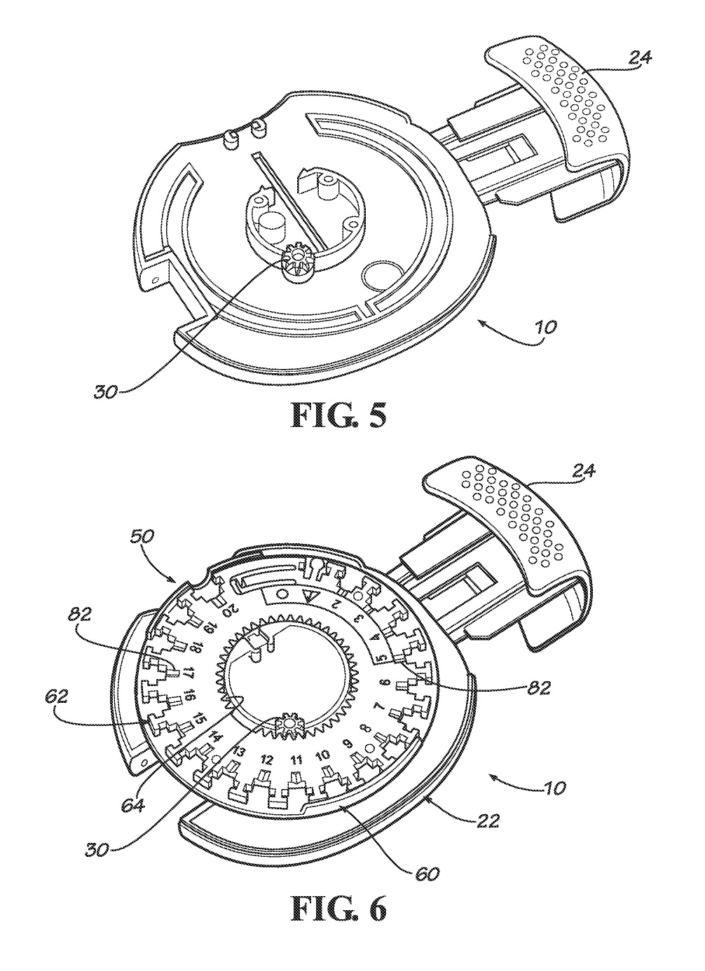

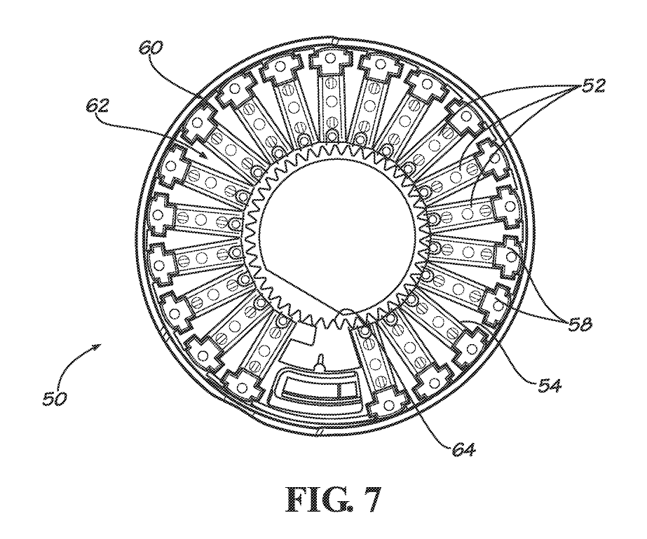

Referring to FIGS. 6-7, the cartridge 50 includes a housing 60 that holds a plurality of the lancets 52, for example, in a radial arrangement on a carrier disk 62. The cartridge 50 includes a cartridge gear 64, for example on the carrier 62, that is engaged and rotationally driven by the pinion output gear 30 of the advancement mechanism 14. In this way, when the advancement mechanism 14 is operated through a first portion (e.g., half) of an operating cycle (e.g., by pulling the operating handle 24 from the retracted to the extended position), the pinion output gear 30 is rotationally driven. The rotating pinion gear 30 in turn rotationally drives the cartridge gear 64 a predetermined indexed angular increment to advance the carrier 62. This in turn advances a used one of the lancets 52 out of the active position and advances a next fresh one of the lancets into the active position for use. The advancement mechanism 14 thereby allows the carrier 62, and thus the lancets 52, to be advanced accurately so that there will be no, or only minimal, issues with misalignment and/or jamming caused by over- or under-advancement into the active position. In typical embodiments, the advancement mechanism 14 also includes an anti-reverse mechanism that functions to prevent the carrier 62 from being reversed to return the used lancet back to the active position during a second portion (e.g., half) of the operating cycle (e.g., by pushing the operating handle 24 from the extended to the retracted position).

Even with a well-designed advancement mechanism 14, on occasion it can slightly over- or under-advance one of the lancets 52 into the active position. For example, the advancement mechanism 14 can be designed to sequentially advance the lancets 52 by 10 degrees, but on occasion over-advance one of the lancets by 0.7 degrees. To minimize jamming and other alignment problems during the rare occasions when the advancement mechanism 14 slightly over- or under-advances one of the lancets 52 into the active position, an alignment mechanism is provided. The alignment mechanism includes at least one register element and at least one spring-biased element that moves between a engaged position and a retracted position. In the engaged position, the spring-biased element matingly engages the register element to better align the active-positioned lancet relative to the cartridge housing. The spring-biased element can be designed so that, in this position, either it is in a relaxed/neutral state or it can still apply a positive biasing force to the register element. And in the retracted position, the spring-biased element does not engage the register element as the lancets are being sequentially advanced into the active position. As such, the alignment mechanism is a mechanical override of the advancement mechanism 14 that fine-tunes the alignment of the lancet 52 in the active position relative to the cartridge housing.

Referring particularly to 8-14, there is shown an alignment mechanism 80 according to an example embodiment of the present invention for use with the lancing device 10 and cartridge 50 disclosed herein. In the depicted embodiment, there are a series of fixed female register elements in the form of recesses 82 defined by the carrier 62 and one spring-biased male element in the form of a protrusion 84 extending from a resilient cantilevered arm 86 defined by the cartridge housing 60.

The recesses 82 and the protrusion 84 have generally conforming female and male shapes, respectively, when viewed from the longitudinal axis of the lancing stroke path. Their shapes are selected so that the protrusion 84 tends to bias into a precise mating engagement in precise alignment with the recess 82 being engaged when the resilient cantilevered arm 86 is in the engaged position. For example, the recesses 82 and the protrusion 84 can be generally wedge-shaped with two symmetrical sides, as in the depicted embodiment. In alternative embodiments, the recesses and the protrusion can have the shape (when viewed along the longitudinal lancing stroke path) of a dome (e.g., a semi-circle), a non-symmetrical triangle/wedge, a cone, a pyramid, a polygon (or portion thereof), or another regular or irregular shape, whether linear, curved, or otherwise shaped. Preferably, the recesses 82 each include an exit ramp surface 83 (e.g., linear, curved, or both) (see FIG. 13) across which a leading surface 85 of the protrusion 84 is driven out of the recesses as the carrier 52 is advanced.

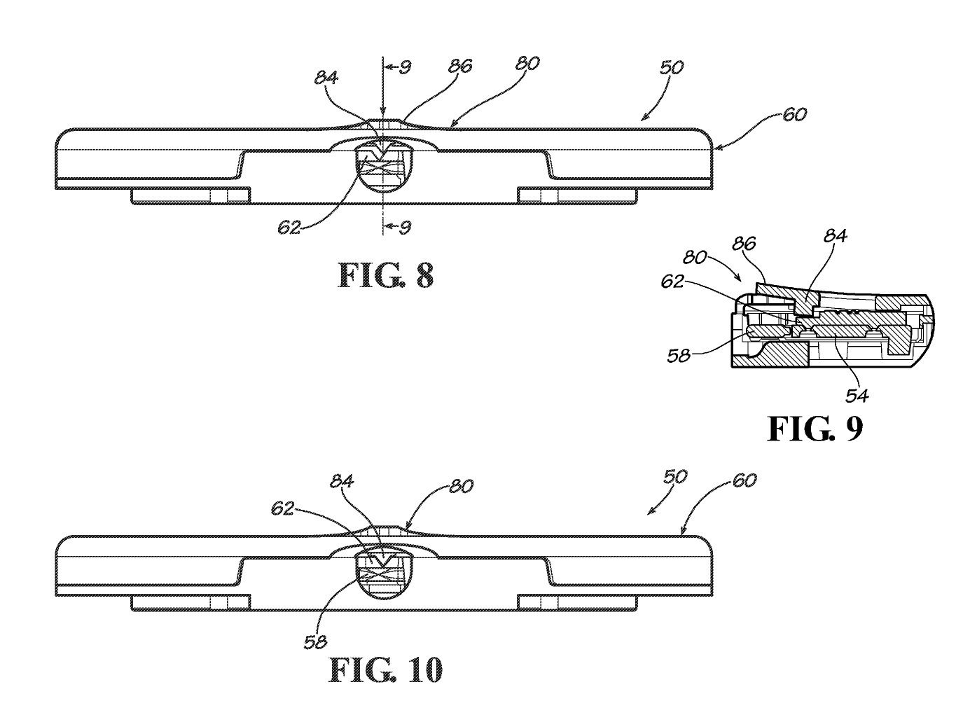

The protrusion 84 is spring-biased toward the engaged position where it sequentially engages each of the recesses 82 of the carrier 62 (as the carrier is advanced by the advancement mechanism) to better align the active-positioned lancet 52 (see FIG. 10), and is able to withdraw from there into the retracted position where it does not engage any of the recesses of the carrier as the lancets are being sequentially advanced (see FIGS. 8-9). The spring-biasing effect can be provided by the protrusion 84 extending transversely from a resilient cantilevered arm 86 that extends from the cartridge housing 60 axially with respect to the lancing stroke path, as in the depicted embodiment. The resilient cantilevered arm 86 can be integrally formed as a part of the cartridge housing 60 and have a generally triangular shape (when viewed from the top), as in the depicted embodiment. In alternative embodiments, the protrusion can be formed on a resilient cantilevered arm that is a separate piece mounted to the cartridge housing and/or be non-axially arranged. And in other alternative embodiments, the protrusion can be spring-biased by another type of spring element such as a compression coil spring, a leaf spring, or another conventional spring element as is known in the art.

In the depicted embodiment, the resilient cantilevered arm 86 is formed in the top wall of the cartridge housing 60 with the protrusion 84 extending downward (inward to within the housing), and the recesses 82 are formed in a top surface of the carrier 62 with each recess aligned with and on the opposite surface of the carrier from a corresponding one of the lancets 52. As each lancet 52 has a corresponding recess 82, the number of recesses included is the same as the number of lancets. In alternative embodiments, the alignment elements (e.g., the recesses) are not located so immediately adjacent their corresponding lancets and instead are formed farther away from their corresponding lancets.

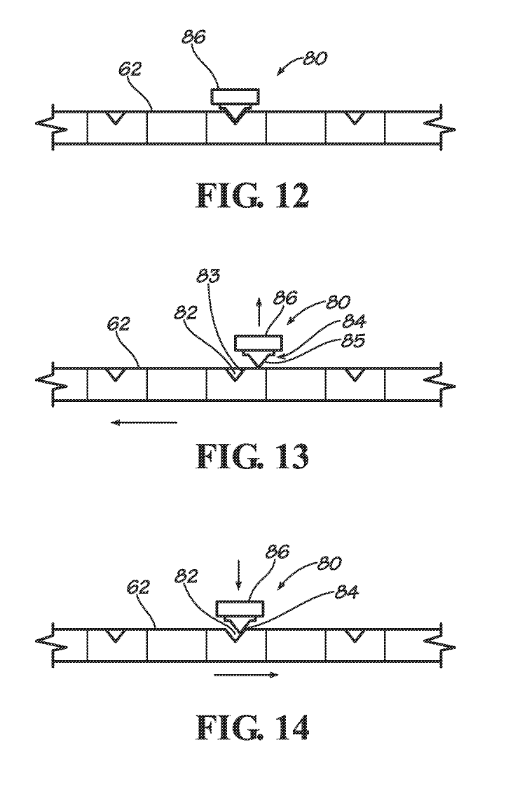

FIGS. 12-14 show the operation of the alignment mechanism 80 of the depicted embodiment. In FIG. 12, the protrusion 84 of the resilient arm 86 is fully inserted into the recess 82 of the carrier 62 to provide precise alignment of the active-positioned lancet 52. Thus, the resilient arm 86 is now in the engaged position. In FIG. 13, the carrier 62 is being advanced (i.e., rotated) by the advancement mechanism (as indicated by the leftward directional arrow). So the protrusion 84 of the resilient arm 86 has been deflected out of engagement with the recess 82 of the carrier 62 (as indicated by the upward directional arrow) so that it rides across the carrier surface as a fresh lancet 52 is being moved into the active position. Thus, the resilient arm 86 is now in the retracted position. And in FIG. 14 (which corresponds to FIG. 8), the carrier 62 has been slightly over-advanced, with the active-positioned lancet 52 not in precise alignment for use. From here, the spring-biasing force of the resilient arm 86 drives the protrusion 84 down (as indicated by the downward directional arrow) into the recess 82, and the mating engagement of the recess by the protrusion causes the carrier 62 to be reversed (as indicated by the rightward directional arrow). In particular, the spring-biasing force of the resilient arm 86 drives the protrusion 84 into the recess 82 until the protrusion again is in full mating engagement with the recess in the engaged position of FIG. 10. As can be seen in FIG. 10, with the protrusion 84 in the engaged position, the position of the carrier 62 has been adjusted so that the active-positioned lancet 52 is now aligned precisely in its intended design position for use.

In the depicted embodiment, the carrier 62 is advanced through a rotary motion to sequentially advance the radially arranged lancets 52 (for begin propelled radially outward) through the rotary motion and into the active position. As such, the recesses 82 (or other register elements) are arranged in a circular shape on the carrier 62. In alternative embodiments, the lancets 52 are generally linearly arranged (e.g., parallel in a longitudinal clip) and sequentially advanced through a generally linear motion into the active position. As such, the recesses 82 (or other register elements) are arranged in a linear arrangement within the cartridge 50 (e.g., on the carrier 62, if one is included in the desired design). In such linear embodiments (and in some rotary embodiments), the advancement mechanism can include rack-and-pinion gearing and/or spring elements for sequentially advancing the lancets to the active position.

In other alternative embodiments, the male/protrusion alignment element and the female/recessed alignment element are reversed in a vice-versa arrangement. That is, the at least one spring-biased element includes a female/recessed surface and the at least one register element is provided by a plurality of male/protrusions. Additionally or alternatively, in some embodiments the at least one spring-biased element is defined by the carrier and the at least one register element is provided by a series thereof defined by the cartridge housing. And additionally or alternatively, in some embodiments the at least one spring-biased element is provided by a series of resilient arms defined for example by the carrier and the at least one register element is provided by a plurality thereof defined by the cartridge or lancing device housing.

In yet other alternative embodiments, instead of at least one of the alignment elements being formed on the carrier, they are formed on the lancets, for example on the lancet bodies or the lancet caps. As this can lead to some frictional issues, the tolerances, spring-biasing forces, and materials for these elements can be selected to account for the longitudinal frictional issues during the lancing stroke. And the recesses can be elongated so that the protrusion does not run into the longitudinal end of the recess and impede the lancet during its lancing stroke.

In still other alternative embodiments, the cartridge does not include a lancet carrier that moves relative to the cartridge housing, and instead the cartridge (and the lancets housed therein) moves relative to the lancing device housing. In such embodiments, the register and spring-biased elements are formed on the cartridge housing (e.g., on its outer surface) and the lancing device housing (e.g., on its inner surface).

And in yet still other alternative embodiments, the alignment mechanism is designed so that it is activated when the actuator (e.g., a button or lever) of the actuation mechanism is activated (e.g., depressed). That is, the alignment elements are brought into mating engagement with each other upon the active-positioned lancet being released to traverse its lancing stroke under the influence of the discharging drive spring. In some such embodiments, the alignment mechanism is cam-operated with a cam pushing one of the alignment elements into engagement with the other one in a timed fashion. In these and related embodiments, the cam and related elements can be integrated into the advancement, charging, and/or actuation mechanism.

In some embodiments, the advancement mechanism mechanically controls the position of the lancet carrier all the way through its advancement to sequentially advance fresh lancets in the active position. In such embodiments, the alignment and/or advancement mechanism includes a release mechanism that disengages the mechanical positioning of the lancets so that they are no longer positively controlled by the advancement mechanism. In addition, persons of ordinary skill in the art will readily recognize that the alignment mechanism can be implemented in cartridge-based lancing devices having numerous other types of advancement mechanisms.

While the invention has been described with reference to preferred and example embodiments, it will be understood by those skilled in the art that a variety of modifications, additions and deletions are within the scope of the invention, as defined by the following claims.

What is claimed is:

1. A cartridge-based device, comprising:
a cartridge including a plurality of items to be used or dispensed for use;
an advancement mechanism adapted to sequentially advance the items of the cartridge to an active position for use; and
an alignment mechanism including at least one register element and at least one spring-biased element that moves between an engaged position and a retracted position, wherein in the engaged position the spring-biased element matingly engages the register element to bias the active-positioned item into a more-precise alignment for use or dispensing, and wherein in the retracted position the spring-biased element does not engage the register element as the items are being sequentially advanced into the active position by the advancement mechanism.

2. The device of claim 1, wherein the at least one register element is provided by a series of register elements and the at least one spring-biased element is provided by a single spring-biased element.

3. The device of claim 2, wherein the register elements are provided by either female or male elements and the spring-biased element includes the other of a male or female element that mates with the register elements.

4. The device of claim 3, wherein the female elements are generally wedge-shaped.

5. The device of claim 3, wherein the female elements each include a ramped exit surface across which the male element rides to bias the spring-biased element to the retracted position.

6. The device of claim 3, wherein the spring-biased element includes a resilient cantilevered arm from which the male element extends.

7. The device of claim 3, wherein the cartridge includes a carrier holding the items and a housing holding the items and the carrier, the register elements are provided by female recesses defined by the carrier, and the spring-biased element is provided by a resilient cantilevered arm that is defined by the cartridge housing and that includes a male protrusion extending therefrom.

8. The device of claim 7, wherein each one of the recesses is positioned adjacent a corresponding one of the items.

9. The device of claim 7, wherein in the retracted position the cantilevered arm deflects away from the carrier and the protrusion rides across the carrier as the items are sequentially advanced into the active position, and wherein in the engaged position one of the items is in the active position and the protrusion is received in one of the recesses corresponding to the active-positioned item.

10. The device of claim 7, wherein the carrier is generally disk-shaped, the items are radially arranged on the carrier and rotationally advanced into the active position, and the recesses are arranged on the carrier in a generally circular shape.

11. A cartridge-based lancing device, comprising:
a cartridge including a housing, a carrier held by the housing, and a plurality of lancets held by the carrier;
an advancement mechanism adapted to sequentially advance the lancets of the cartridge to an active position for use; and
an alignment mechanism including a series of register elements and a spring-biased element that moves between an engaged position and a retracted position, wherein the register elements and the spring-biased element have mating shapes, wherein the register elements are provided by either female or male elements and the spring-biased element includes the other of a female or male element, wherein in the engaged position the spring-biased element matingly engages one of the register elements to bias the active-positioned lancet into a more-precise alignment for use, wherein in the retracted position the spring-biased element does not engage any of the register elements as the lancets are being sequentially advanced into the active position by the advancement mechanism, and wherein the alignment mechanism mechanically overrides the advancement mechanism to fine-tune the alignment of the active-positioned lancet.

12. The device of claim 11, wherein the female elements are generally wedge-shaped and each include a ramped exit surface across which the male element rides to bias the spring-biased element to the retracted position.

13. The device of claim 11, wherein the spring-biased element includes a resilient cantilevered arm from which the male or female element extends.

14. The device of claim 11, wherein the register elements are provided by female recesses defined by the carrier, the spring-biased element is provided by a resilient cantilevered arm that is defined by the cartridge housing and that includes a male protrusion extending therefrom, and in the retracted position the cantilevered arm deflects away from the carrier and the protrusion rides across the carrier as the lancets are sequentially advanced into the active position, and wherein in the engaged position one of the lancets is in the active position and the protrusion is received in one of the recesses corresponding to the active-positioned lancet.

15. The device of claim 14, wherein each one of the recesses is positioned adjacent a corresponding one of the lancets.

16. The device of claim 11, wherein the carrier is generally disk-shaped, the lancets are radially arranged on the carrier and rotationally advanced into the active position, and the recesses are arranged on the carrier in a generally circular shape.

17. A multi-lancet cartridge for use with a lancing device having an advancement mechanism, the multi-lancet cartridge comprising:
a housing;
a carrier held by the housing;
a plurality of lancets held by the carrier and sequentially advanceable by the advancement mechanism to an active position for use; and
an alignment mechanism including a series of register elements and a spring-biased element that moves between an engaged position and a retracted position, wherein the register elements and the spring-biased element have mating shapes, wherein the register elements are provided by either female or male elements and the spring-biased element includes the other of a female or male element, wherein in the engaged position the spring-biased element matingly engages one of the register elements to bias the active-positioned lancet into a more-precise alignment for use, wherein in the retracted position the spring-biased element does not engage any of the register elements as the lancets are being sequentially advanced into the active position by the advancement mechanism, and wherein the alignment mechanism mechanically overrides the advancement mechanism to fine-tune the alignment of the active-positioned lancet.

18. The multi-lancet cartridge of claim 17, wherein the female elements are generally wedge-shaped and each include a ramped exit surface across which the male element rides to bias the spring-biased element to the retracted position.

19. The multi-lancet cartridge of claim 17, wherein the spring-biased element includes a resilient cantilevered arm from which the male or female element extends.

20. The multi-lancet cartridge of claim 17, wherein the register elements are provided by female recesses defined by the carrier, the spring-biased element is provided by a resilient cantilevered arm that is defined by the cartridge housing and that includes a male protrusion extending therefrom, and in the retracted position the cantilevered arm deflects away from the carrier and the protrusion rides across the carrier as the lancets are sequentially advanced into the active position, and wherein in the engaged position one of the lancets is in the active position and the protrusion is received in one of the recesses corresponding to the active-positioned lancet.

21. The multi-lancet cartridge of claim 20, wherein each one of the recesses is positioned adjacent a corresponding one of the lancets.

22. The multi-lancet cartridge of claim 17, wherein the carrier is generally disk-shaped, the lancets are radially arranged on the carrier and rotationally advanced into the active position, and the recesses are arranged on the carrier in a generally circular shape.
